(19) United States
(12) Patent Application Publication
(10) Pub. No.: US 2026/0072041 A1
(43) Pub. Date: Mar. 12, 2026

(54) Disposable Indicator

(71) Applicant: Camille Sylvestre, Suffield, CT (US)

(72) Inventor: Camille Sylvestre, Suffield, CT (US)

(73) Assignee: CKNS, LLC, Suffield, CT (US)

(21) Appl. No.: 18/826,335

(22) Filed: Sep. 6, 2024

(51) Int. Cl. G01N 33/68

(52) U.S. Cl. CPC G01N 33/689; G01N 2333/471

(57) ABSTRACT

The present disclosure provides a disposable indicator for identifying detectable attributes. The system may comprise a front material, a back material opposite of the front material, and an absorbent material between the front material and the back material. The front material may include a reactant region and a control region, wherein both regions may be configured to receive fluids, namely, bodily discharge. The reactant region may be configured to receive a reactant, wherein the reactant mixes with received bodily discharge to detect the presence of detectable attributes. The control region may be configured to react when fluid may be detected, wherein the control region confirms the effectiveness of the disposable indicator. The present disclosure provides a method for an indicator for identifying detectable attributes, wherein the disposable indicator may be placed on a user to receive and detect the presence of detectable attributes in the user's bodily discharge.

### BACKGROUND

Bodily discharge—such as urine, vaginal secretions, and anal discharge—can serve as a critical indicator of a wide range of biological and chemical conditions. These secretions may carry measurable biomarkers associated with infection, disease states, hormonal changes, reproductive cycles, and other health-related phenomena. However, such discharge often goes unnoticed or may be challenging to interpret without specialized tools, delaying diagnosis or necessary intervention.

In particular, detectable attributes in bodily discharge may include, but are not limited to, pH levels, protein markers, hormonal concentrations, and microbial indicators. For example, luteinizing hormone (LH) surges in urine are a key biomarker of ovulation. Existing technologies—such as ovulation pee strips, digital hormone monitors, and basal body temperature charting—can be complex, costly, or cumbersome to interpret, which limits accessibility and consistent use. A simplified, integrated detection method could improve fertility tracking and reproductive health monitoring.

As a second example, urinary tract infections (UTIs) are among the most common bacterial infections, particularly in individuals with female anatomy. Early signs of a UTI—such as the presence of nitrites (produced by certain bacteria) and leukocytes (white blood cells indicating immune response)—can be detected in urine. However, many individuals may experience nonspecific symptoms or attribute discomfort to other causes, delaying care. Current UTI tests are often performed in clinical settings or rely on over-the-counter strips that require interpretation and are not integrated into daily hygiene routines. A system that automatically detects UTI indicators in bodily discharge could enable earlier intervention, reducing the risk of kidney infections or systemic complications.

Current devices on the market are largely limited to rudimentary moisture detection, which may alert the user to the presence of fluid but cannot distinguish between various biological fluids or the presence of clinically relevant biomarkers. These limitations significantly reduce the diagnostic utility of such products.

What is needed, therefore, is a device or method capable of detecting specific biochemical or hormonal attributes in bodily discharge—such as those found in urine, vaginal, or anal secretions—in a fast, non-invasive, and user-friendly manner. This advancement would allow individuals to distinguish between different types of discharge and understand whether a physiological event or condition (e.g., ovulation, infection, pregnancy complications) is occurring, prompting timely medical action when necessary.

The ability to rapidly identify and interpret these specific indicators—without reliance on complex instrumentation or manual charting—would represent a significant improvement over current discharge detection technologies. In certain embodiments, this solution may involve integrated test materials or colorimetric indicators that react to the presence of a particular analyte, such as LH in urine or nitrites indicative of a UTI, thus providing real-time feedback to the user regarding their reproductive or urological health.

### DETAILED DESCRIPTION

The present disclosure provides generally for a disposable indicator system for detecting one or more targeted detectable attributes in bodily discharge. According to the present disclosure, the disposable indicator may serve as a diagnostic support tool for enhancing early detection of various health conditions—such as hormonal fluctuations, infections, medical conditions and reproductive events—by identifying specific biomarkers or chemical properties present in urinary, vaginal, or anal secretions.

The system is designed to be used by both laypersons in home or personal environments, and by healthcare professionals in clinical, hospital, or diagnostic settings. In consumer use, the disposable indicator provides an accessible, non-invasive, and intuitive method for individuals to monitor key aspects of their health—such as ovulation timing or the onset of urinary tract infections—without the need for complex equipment, charting, or specialized knowledge. In professional or institutional healthcare settings, the same device may be utilized as a preliminary screening tool or as part of routine patient monitoring to detect early indicators of infection, inflammation, or reproductive status.

A disposable indicator for detecting targeted detectable attributes may alert a user or care provider to the presence of a relevant physiological or pathological condition, thereby prompting timely health decisions or medical intervention. By integrating diagnostic capability into an easy-to-use, hygienic, and disposable form factor, this system addresses the limitations of existing solutions that are either overly simplistic (e.g., moisture-only detection) or too complex for widespread daily use.

In the following sections, detailed descriptions of examples and methods of the disclosure will be provided. The description of both preferred and alternative embodiments, though thorough, is exemplary only. It will be understood by those skilled in the art that variations, modifications, and alterations may be apparent. Therefore, the examples provided do not limit the scope of the underlying disclosure as defined by the claims.

### Glossary

Reactant: As used herein, refers to any substance or chemical agent that reacts with one or more detectable attributes in bodily discharge to produce a visible, measurable, or otherwise perceptible indication.

Control: As used herein, refers to any portion of the disposable indicator that serves as a reference to verify the proper function of the test. The control may be a portion of the indicator that is non-reactive to targeted detectable attributes but provides assurance that the indicator is active and functioning.

Disposable Indicator: As used herein, refers to any material, component, or device designed for single or limited use that incorporates a reactant and control to test bodily discharge for targeted detectable attributes. In some aspects, the disposable indicator may be an insert or integrated portion of undergarments, adult diapers, or hygiene products. In other embodiments, the disposable indicator may be incorporated into care materials used in clinical, nursing home, rehabilitation, or home-care environments. By way of example and not limitation, the disposable indicator may include maxi pads, pee pads, panty liners, tampons, diapers, chucks, underwear, or towels. The indicator is suitable for layperson, caregiver, or professional use and is especially advantageous for nonverbal or cognitively impaired individuals who cannot report symptoms directly.

Indicator: As used herein, refers to any visual or otherwise perceptible result generated by the interaction of a bodily discharge with the reactant and/or control, signaling the presence or absence of one or more targeted detectable attributes.

Detectable Attributes: As used herein, refers to any biological, chemical, or hormonal marker that may be present in bodily discharge and that can be identified through interaction with a configured reactant. Non-limiting examples include: luteinizing hormone (LH), nitrites, leukocytes, pH level changes, yeast, candida, prostate-specific antigen (PSA), mycoplasma, alpha-fetoprotein, amniotic fluid, bacterial vaginosis, glucose, red and white blood cells, ketones, hemoglobin, bilirubin, sexually transmitted infections (STIs), trichomoniasis, and drug presence.

Targeted Detectable Attributes: As used herein, refers to specific detectable attributes that the disposable indicator is designed to identify. These attributes are preselected and targeted through the choice of reactant(s) used in the device. Targeted detectable attributes do not include wetness or moisture alone as an indicator.

Bodily Discharge: As used herein, refers to any fluid secreted by the human body, including but not limited to urinary, vaginal, and anal secretions. Non-limiting examples of bodily discharge include urine, feces, blood, saliva, semen, plasma, amniotic fluid, and vaginal secretions.

Care Environment: As used herein, refers to any location or context where health monitoring or hygiene care occurs, including homes, medical clinics, hospitals, urgent care centers, assisted living facilities, nursing homes, rehabilitation centers, or any setting in which an individual—especially one who may be nonverbal, bedridden, developmentally delayed, or cognitively impaired—receives medical attention or caregiver support.

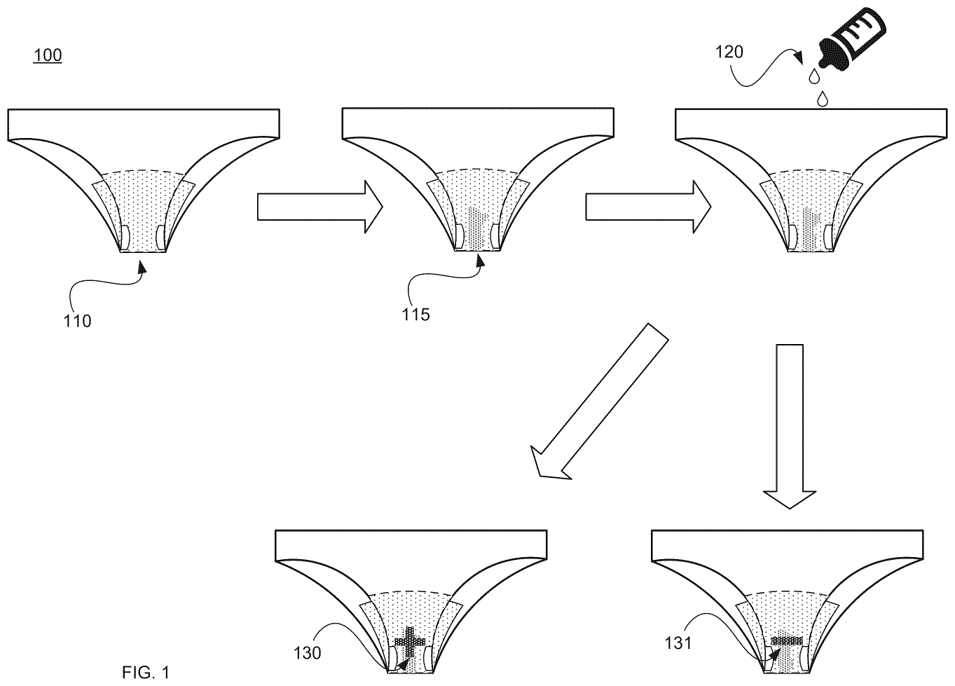

Referring now to FIG. 1, a process flow 100 for using a disposable indicator 110, according to some embodiments of the present disclosure, is illustrated. In some aspects, the disposable indicator 110 may receive bodily discharge 115. Reactant 120 may be added to the bodily discharge 115 to indicate whether the contents include detectable attributes wherein the disposable indicator may be configured to provide information regarding their health or condition. An indicator 130, 131 may clearly show a positive result indication 130 or a negative result indication 131.

In some embodiments, the reactant 120 may be configured to chemically react with the bodily discharge 115 and the indicator 130, 131, wherein the result of the interaction may reveal whether the bodily discharge 115 includes detectable attributes. By way of example and not limitation, the reactant 120 and indicator 130, 131 may be configured to detect the presence of at least one targeted detectable attribute.

In some embodiments, the reactant 120 may be configured to test for the presence of at least one targeted detectable attribute. In some implementations, the confirmed presence of at least one targeted detectable attribute may show a positive result indication 130 on the disposable indicator 110 whereas the confirmed absence of the at least one targeted detectable attribute may show a negative result indicator 131 on the disposable indicator 110. In some aspects, the disposable indicator 110 may be configured to identify at least one targeted detectable attribute, wherein one disposable indicator may be configured to detect the presence of luteinizing hormone, whereas a different disposable indicator may be configured to detect the presence of urinary tract infection (UTI), as a non-limiting example.

In some aspects, the detectable attributes may include different hormonal profiles and may include a predefined threshold of estrogen, human chorionic gonadotrophin (hCG), Estriol, or Inhibin-A in the bodily discharge 115, as non-limiting examples. In some implementations, the disposable indicator 110 may be configured to detect a variety of detectable attributes. By way of example and not limitation, the detectable attributes may include the presence of infections, cervical health, vaginal health, allergies, and immune system status in bodily discharge. In some aspects, the detectable attributes may include yeast infections, urinary tract infections (UTIs), or sexually transmitted infections (STIs), wherein the disposable indicator 110 may be configured to notify a user whether an infection may be present. In some embodiments, the detectable attributes may be configured to determine cervical or vaginal health data. By way of example and not limitation, the reactant 120 and indicator 130, 131 may be configured to react to bodily discharge that may be symptomatic of cancer or other disease.

In some aspects, the detectable attributes may include infection, allergy, or chemical reactions, wherein the reactant 120 and indicator 130, 131 may be configured to notify a user if they may be experiencing allergic or chemical reactions. In some implementations, the disposable indicator 110 may be used by a user to confirm whether or not they are experiencing an allergic reaction to a specific substance, material, or product. In some embodiments, the disposable indicator 110 may be configured to detect a variety of detectable attributes simultaneously, wherein the indicator 130, 131 may be configured to provide unique notifications depending on the specific type of detectable attribute identified. In some aspects, the disposable indicator 110 may be configured to detect a variety of detectable attributes from secretions or discharge from genitalia or the anus. In some implementations, the disposable indicator 110 may be configured to detect the presence of infection or disease.

By way of example and not limitation, the disposable indicator may be configured to receive vaginal discharge, wherein the vaginal discharge may include cervical mucus. In some aspects, the at least one targeted detectable attribute may include luteinizing hormone. In some aspects, the presence of luteinizing hormone may indicate that a user is ovulating. In some implementations, the at least one targeted detectable attribute may include monoclonal luteinizing hormone antibody, protein and other profiles, wherein the disposable indicator may inform the user of the details of their menstrual cycle and fertile window. In some aspects, the disposable indicator may provide information on when to time intercourse for conception. In some implementations, the at least one targeted detectable attribute may include bacteria in addition to monoclonal luteinizing hormone antibody, as non-limiting examples, wherein the disposable indicator may simultaneously provide information on ovulation and on whether the user may have a urinary tract infection (UTI).

By way of example and not limitation, the disposable indicator may be used by a cardiovascular patient, wherein the at least one targeted detectable attribute may include cholesterol, triglycerides, and troponin, as non-limiting examples. In some aspects, the disposable indicator may receive bodily discharge, wherein the presence of the at least one targeted detectable attribute may inform the user on their cardiovascular condition. In some implementations, the at least one targeted detectable attribute may include a single detectable attribute whereas, in other implementations the at least one targeted detectable attribute may include multiple detectable attributes.

By way of example and not limitation, the disposable indicator may be configured to receive urine. In some aspects, the reactant may be configured to react to the at least one targeted detectable attribute that may be present in a urine sample. For example, the at least one targeted detectable attribute may include UTI, STI, or drug detection, as non-limiting examples. In some implementations, the reactant may be configured to react with a single at least one targeted detectable attribute, whereas in some implementations the reactant may be configured to react with multiple at least one detectable attribute. In some embodiments, the reactant may simultaneously identify whether a user may have a UTI, STI, or drugs in their system.

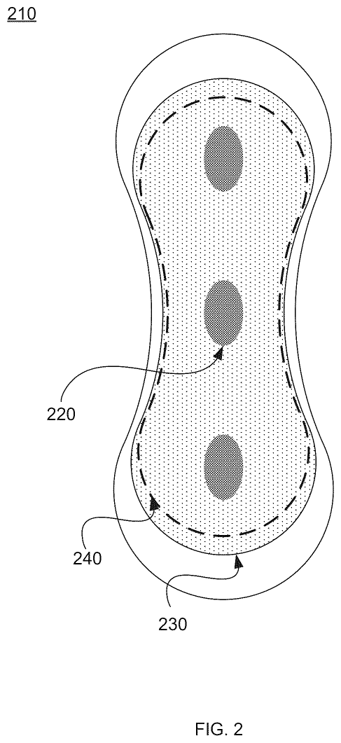

Referring now to FIG. 2, a disposable indicator with reactant pods for detecting bodily discharge for detectable attributes, according to some embodiments of the present disclosure, is illustrated. In some embodiments, the indicator 210 may include a front material that may include reactant pods 220, reactant regions 240 and control regions 230. In some implementations, the reactant regions 240 may include an indicator configured to interact with a reactant and bodily discharge to provide information regarding their health or condition. In some aspects, the reactant pods 220 may include the reactant, wherein the reactant pods 220 may be configured to release the reactant onto the reaction regions 240. By way of example and not limitation, the reactant pods 220 may be configured to dissolve in contact with fluid, wherein the dissolving of the reactant pods 220 may release the reactant into the reactant regions 240.

In some implementations, the indicator, through the reaction between the reactant and the bodily discharge, may determine the presence of at least one targeted detectable attribute. In some aspects, the reactant may be configured to interact with different hormonal profiles or alpha-fetoprotein. As a way of example and not limitation, the reactant regions 240 may be configured to change color or change pattern in the presence of at least one targeted detectable attribute. In some aspects, the control regions 230 may be configured to change color upon absorbing fluid, wherein the control regions 230 may indicate whether the disposable indicator 210 may be effective.

In some embodiments, the disposable indicator 210 may include a back material opposite of the front material, wherein the back material may include adhesive 250. In some aspects, the adhesive 250 may be configured to attach the disposable indicator 210 to a clothing material. By way of example and not limitation, the clothing material may include undergarments or underwear. In some embodiments, the material between the front material and the back material may prevent any fluids from touching the clothing material. By way of example and not limitation, the disposable indicator 210 may be configured as underwear, undergarments, panty liners, maxi pads, and diapers.

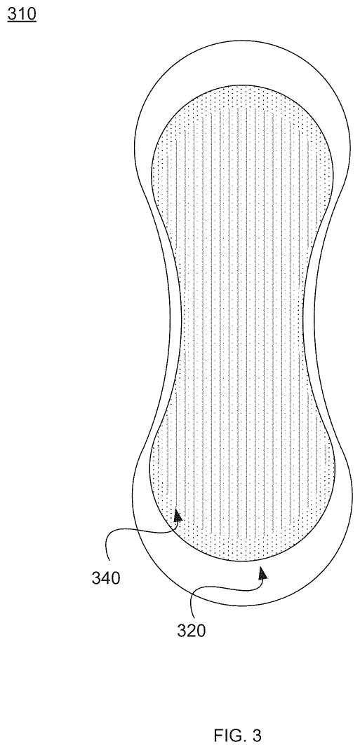

Referring now to FIG. 3, a disposable indicator for detecting detectable attributes, according to some embodiments of the present disclosure, is illustrated. In some embodiments, the disposable indicator 310 may include reactant regions 340 and control regions 320. In some aspects, the reactant regions 340 may be configured to receive bodily discharge, wherein reactants within the reactant regions 340 may determine whether the bodily discharge may include detectable attributes. In some embodiments, the reactant regions 340 may alert the user of a condition requiring medical attention. As a way of example and not limitation, the reactant regions 340 may be configured to change color or change pattern in the presence of at least one targeted detectable attribute.

In some aspects, the reactant regions 340 may be organized in lines that continue vertically across the length of the disposable indicator 310. In some implementations, the indicator, through the reaction between the reactant and the bodily discharge, may determine the presence of detectable attributes. In some aspects, the reactant may be configured to interact with different hormonal profiles, such as monoclonal luteinizing hormone antibodies. By way of example and not limitation, the reactant regions 340 may be configured to indicate whether the reactant detected luteinizing hormone (LH), nitrites, leukocytes, pH level changes, yeast, candida, prostate-specific antigen (PSA), mycoplasma, alpha-fetoprotein, amniotic fluid, bacterial vaginosis, glucose, red and white blood cells, ketones, hemoglobin, bilirubin, sexually transmitted infections (STIs), trichomoniasis, drug presence or other detectable attributes through a changing of color or pattern. In some implementations, the area between each reactant region 340 may include a control region 320, wherein the control region 320 may be configured to change color or pattern when it absorbs fluid. In some aspects, the control regions 320 may be configured to indicate whether the disposable indicator 310 may be effective.

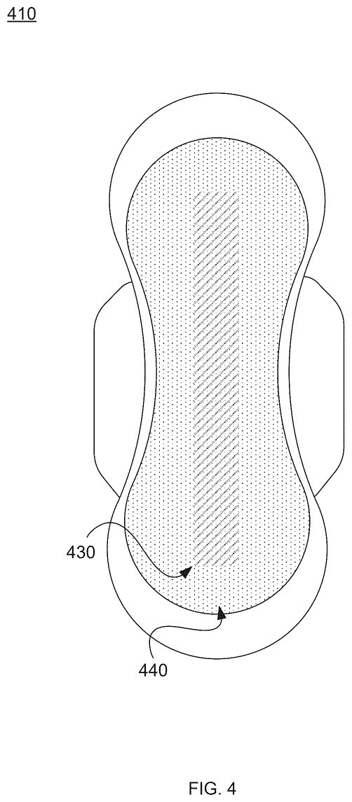

Referring now to FIG. 4, a disposable indicator for identifying detectable attributes, according to some embodiments of the present disclosure is illustrated. In some embodiments, the disposable indicator may include a front material and a back material opposite of the front material. In some aspects, the front material of the disposable indicator may include a reaction region 430 and a control region 440. In some aspects, the reaction region 430 may include a reactant configured to detect detectable attributes. In some implementations, the reaction region 430 may receive bodily discharge from a user, wherein the reaction region 430 may indicate the presence of luteinizing hormone, bacteria or other detectable attributes by changing color or pattern, as non-limiting examples.

In some implementations, the reaction region 430 may be vertical line that moves length wise across the disposable indicator 410, wherein the area surrounding the reaction region 430 may be the control region 440. In some aspects, the control region 440 may be configured to change color or pattern, as non-limiting examples, when exposed to fluid to ensure that the disposable indicator may be effective. In some embodiments, the reaction region 430 may include a gel, strip, or chemical that may be configured to detect the presence of detectable attributes.

In some embodiments, the disposable indicator 410 may include a back material opposite of the front material, wherein the back material includes an adhesive 420. In some aspects, the adhesive 420 may be wings, wherein the wings may be configured to wrap around a clothing material and stick. In some implementations, the adhesive 420 may face the back material, wherein folding the adhesive 420 over the clothing material may attach it and prevent further movement, wherein a user may go about their day to day without having to reattach or readjust the disposable indicator 410.

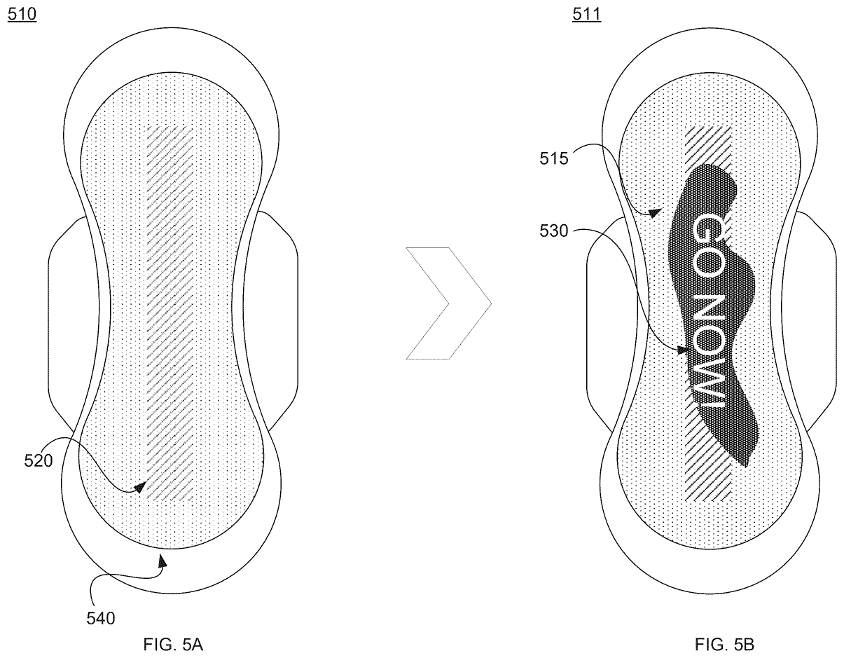

Now referring to FIG. 5A, an indicator flow for detecting bodily discharge for detectable attributes, according to some embodiments of the present disclosure is illustrated. In some embodiments, the disposable indicator 510 may include a front material and a back material opposite of the front material. In some aspects, the front material of the disposable indicator may include a reaction region 520 and a control region 540. In some aspects, the reaction region 520 may include a reactant configured to detect detectable attributes. In some implementations, the reaction region 520 may receive bodily discharge from a user, wherein the reaction region 520 may indicate the presence of or other detectable attributes, by changing color or pattern, as non-limiting examples.

In some implementations, the reaction region 520 may be vertical line that moves length wise across the disposable indicator 510, wherein the area surrounding the reaction region 520 may be the control region 540. In some aspects, the control region 540 may be configured to change color or pattern, as non-limiting examples, when exposed to fluid to ensure that the disposable indicator may be effective. In some embodiments, the reaction region 520 may include a gel, strip, or chemical that may be configured to detect the presence of luteinizing hormone, bacteria or other detectable attributes by changing color or pattern, as non-limiting examples.

Now referring to FIG. 5B, an indicator flow for detecting bodily discharge for detectable attributes, according to some embodiments of the present disclosure is illustrated. In some embodiments, the disposable indicator 511 may receive and absorb bodily discharge from a user. In some implementations, the bodily discharge may be absorbed by the reactant region 530 and the control region 515. In some aspects, the control region 515 may be configured to change color or pattern, as non-limiting examples, when exposed to fluid to ensure that the disposable indicator may be effective.

In some embodiments, the reactant region 530 may be configured to show a positive indicator result or a negative indicator result, wherein the positive indicator result may be revealed in the presence of detectable fluids. In some implementations, the reactant of the reactant region 530 may react with vaginal secretions or other detectable fluids to produce a positive indicator result. In some aspects, the positive indicator result may reveal an alert 550. As a way of example and not limitation, the alert 550 may be display a message to the user. For example, if the at least one targeted detectable fluid may be urine, the message may read “UTI!” to prompt the user that they have bacteria which notifies them of a urinary tract infection (UTI). In some aspects, a negative indicator result may produce no alert, wherein the reactant region 530 does not have a visible change when in contact with the bodily discharge. In some implementations, the control region 515 may be configured to change color or pattern, as non-limiting examples, regardless of whether the bodily discharge may include detectable attributes.

### Conclusion

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described components and systems can generally be integrated together in a single product or packaged into multiple products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

Reference in this specification to “one embodiment,” “an embodiment,” any other phrase mentioning the word “embodiment”, “aspect”, or “implementation” means that a particular feature, structure, or characteristic described in connection with the embodiment is included in at least one embodiment of the disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional.

Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

1. A disposable indicator for identifying detectable attributes including:
a reactant configured to detect at least one targeted detectable attribute when mixed with vaginal discharge, urinary discharge, or anal discharge, wherein the at least one targeted detectable attribute is amniotic fluid or alpha fetal protein;
a front material including:
a reactant region with an indicator, wherein the reactant region displays a positive result indication in the presence of the at least one targeted detectable attribute when combined with the reactant;
a control region that surrounds the reactant region, wherein the control region is configured to react when the fluid is detected, wherein the control region is confirming the disposable indicator is effective;
a back material opposite of the front material, wherein the back material includes an adhesive configured to attach the disposable indicator to a clothing material; and
an absorbent material between the front and back material of the disposable indicator configured to absorb the fluid.

2. (canceled)

3. (canceled)

4. (canceled)

5. The disposable indicator of claim 1, wherein the positive result indication is configured to change a color or pattern of the reactant region.

6. The disposable indicator of claim 1, wherein one or both the front material and the absorbent material are impregnated with the reactant.

7. The disposable indicator of claim 1, wherein a predetermined pattern or color is configured to a negative result indication.

8. The disposable indicator of claim 1, wherein the reactant region is a vertical strip positioned lengthwise across the disposable indicator.

9. The disposable indicator of claim 7, wherein the reactant region changes color in the presence of the at least one targeted detectable attribute and the control region changes color in the presence of fluid.

10. The disposable indicator of claim 7, wherein the presence of the at least one targeted detectable attribute in the reactant region reveals an alert in typographical characters.

11. The disposable indicator of claim 1, wherein the reactant region further includes reactant pods that are configured to dissolve or open in the presence of fluid, wherein the dissolved or opened reactant pods release reactant into the reactant region.

12. The disposable indicator of claim 1, wherein the reactant region includes a plurality of vertical lines that are positioned lengthwise across the disposable indicator, wherein the control region is the area between each of the plurality of lines of the reactant region.

13. The disposable indicator of claim 1, wherein the reactant is a separate solution, wherein the reactant is applied to the reactant region after fluid is received.

14. The disposable indicator of claim 1, wherein the disposable indicator includes clothing material, wherein the disposable indicator is configured to serve as underwear, undergarments, panty liners, maxi pads, and diapers.

15. A method for determining presence of detectable attributes with a disposable indicator including:
placing the disposable indicator in a position that is configured to receive bodily discharge, wherein the disposable indicator includes a reactant region with an indicator and a control region with an indicator that is configured to determine whether the disposable indicator is effective by showing a consistent result when the disposable indicator is met with fluid.
receiving bodily discharge on the disposable indicator, wherein the bodily discharge is received by both the reactant region and the control region;
mixing the bodily discharge with a reactant at the reaction region, wherein the indicator of the reactant is configured to determine if at least one targeted detectable attribute is present in the bodily discharge;
providing an indication as to whether the at least one targeted detectable attribute is present in the bodily discharge.

16. The method of claim 15, wherein the at least one targeted detectable attribute comprises amniotic fluid.

17. The method of claim 15, wherein the indicator on the reactant region is configured to display a positive result indicator or a negative result indicator, wherein a positive result indicator is displayed in the form of a color change or pattern change of the reactant region.

18. The method of claim 15, wherein the reactant region includes reactant pods that are configured to dissolve in the presence of fluid, wherein the reactant pods release reactant to determine whether the fluid contains at least one targeted detectable attribute.

19. The method of claim 15, wherein the method includes pouring a predetermined amount of reactant onto the reactant region when the disposable indicator is exposed to bodily discharge, wherein the reactant detects the presence of at least one detectable attribute.

20. The method of claim 15, wherein the reactant is configured to detect alpha-fetoprotein.
